(19) United States
(12) Patent Application Publication
(10) Pub. No.: US 2018/0360810 A1
(43) Pub. Date: Dec. 20, 2018

(54) NUPHARIDINE COMPOUNDS AND DERIVATIVES THEREOF FOR THE TREATMENT OF CYSTEINE PROTEASE RELATED DISEASES

(71) Applicants: B. G. NEGEV TECHNOLOGIES AND APPLICATIONS LTD., AT BEN-GURION UNIVERSITY, Beer Sheva (IL); MOR RESEARCH APPLICATIONS LTD., Tel Aviv (IL)

(72) Inventors: Avi GOLAN-GOLDHIRSH, Midreshet Ben-Gurion (IL); Jacob GOPAS, Beer Sheva (IL); Udi ZURGIL, Kibbutz Sede Boqer (IL)

(21) Appl. No.: 16/060,377

(22) PCT Filed: Dec. 8, 2016
(86) PCT No.: PCT/IL2016/051316

§ 371 Date: Jun. 7, 2018

Related U.S. Application Data
(60) Provisional application No. 62/264,474, filed on Dec. 8, 2015.

(51) Int. Cl. A61K 31/438; A61K 31/4375; A61K 36/62

(52) U.S. Cl. CPC A61K 31/438; A61P 35/00; A61K 36/62; A61K 31/4375

(57) ABSTRACT

Disclosed a method of treating a disease or a disorder characterized by protease activity. A pharmaceutical composition for treating a disease or a disorder characterized by protease activity is further disclosed. A method of detecting a cysteine protease from a biological sample is also disclosed.

This application claims the benefit of priority from U.S. Provisional Patent Application No. 62/264,474 filed on Dec. 8, 2015. The content of the above document is incorporated by reference in its entirety as if fully set forth herein.

### FIELD OF THE INVENTION

The present invention, in some embodiments thereof, relates to compounds derived from nupharidine and synthetic derivatives thereof for the treatment of a disease or a disorder characterized by protease activity.

### BACKGROUND OF THE INVENTION

Various plant organs of Nuphar lutea (L.) SM. (Nymphaeaceae) are used in traditional medicine. For example, the use of Nuphar lutea extracts for medicinal purposes by aboriginals of the Canadian boreal forest was reported by Uprety et al. (Journal of Ethnobiology and Ethnomedicine 8, 7, 2012).

Proteases are proteolytic enzymes, which catalyze the hydrolysis of peptide bonds in all living organisms. Proteases were reported in plants, bacteria, protozoa, fungi, parasitic organisms and mammals, and are essential for a myriad of biochemical and metabolic processes in living cells.

Proteases are designated either as endo- or exopeptidases, and cleave peptide bonds within a protein or peptide and remove amino acids from the N- or the C-terminus, respectively. Depending on the catalytic residue responsible for peptide hydrolysis, the proteases have been divided into serine, cysteine, aspartic, and metallo-proteases. Due to the growing number of proteases which are being discovered, a more in depth classification has become necessary.

Barrett and Rawlings (2001) have established a classification system, which organizes the various proteases within their individual catalytic category into evolutionary families and clans, and which forms a comprehensive and continuously expanding catalog of proteases: the MEROPS database. An individual protease is assigned to a particular clan based on the three-dimensional structure, the arrangement of catalytic residues in the polypeptide chains and limited similarities in amino acid sequence around the catalytic amino acid residues.

Proteases are involved in several disease states and thus have gained much attention as therapeutic targets with regards to viral and parasitic infections, stroke, cancer, Alzheimer's disease, neuronal cell death, and arthritis.

U.S. Patent Application 20130122114 provides therapeutic methods which include inhibition of nuclear factor κb pathway in a cell based on the discovery of an active fraction of a plant extract termed NUP or a composition which includes NUP. NUP is used in treating and managing different diseases such as cancer, inflammation, and virus infections.

### SUMMARY OF THE INVENTION

The present invention, in some embodiments thereof, relates to compounds derived from nupharidine and synthetic derivatives thereof for the treatment of a disease or a disorder characterized by protease activity.

According to an aspect of some embodiments of the present invention, there is provided a method for treating a disease or a disorder characterized by protease activity or, in some embodiments, elevated cysteine protease activity, in a subject in need thereof, the method comprising administering to the subject a pharmaceutically effective amount of a compound having the general formula I:

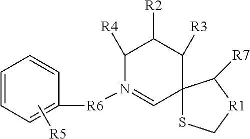

wherein:

R1 is CH₂, C₂-C₄ alkyl or is represented by the structure of Formula II:

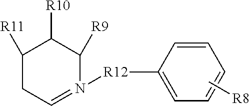

such that Formula I is in the form of Formula Ia:

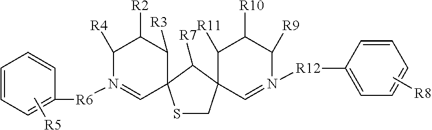

wherein:

R2, R3, R4, R7, R9, R10, R11 are independently selected at each occurrence from hydrogen or C1-C8-alkyl optionally substituted by hydroxyl;

R6 and R12 are independently C1-C8 alkyl;

each R5 and R8 represents 0, 1, 2, 3, 4, or 5 substitutents independently selected at each occurrence from the group consisting of: hydroxy, halogen, cyano, C₁-C₄ alkyl, C₁-C₄ alkoxy, C₁-C₄ haloalkyl, C₁-C₄ haloalkoxy, —SO₂—R¹³, —P(O)(OR¹⁴)(OR¹⁵), —C(O)—NR¹⁴R¹⁵, —N(CH₃)—CO—O—(C₁-C₄) alkyl, —NH—CO—O—(C₁-C₄) alkyl, —NH—CO—(C₁-C₄)alkyl, —N(CH₃)—CO—(C₁-C₄) alkyl, —NH—(CH₂)₂—OH;

R¹³ is selected from: C₁-C₄ alkyl, NH₂, or —(CH₂)₂—OH;

R¹⁴ is selected from: hydrogen or C₁-C₄ alkyl; and

R¹⁵ is selected from: hydrogen, C₁-C₄ alkyl, —(CH₂)₂—OH, —(CH₂)₂—O—CH₃, —(CH₂)₃—OH, —(CH₂)₃—O—CH₃, 3-oxetany.

In some embodiments, R1 is CH₂, R5 represents 0 substituent, and R2, R3, R4, and R7 are each hydrogens.

In some embodiments, the compound is represented by the structure of formula III:

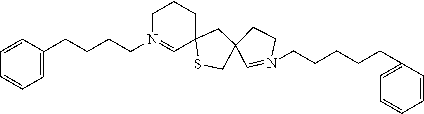

According to an aspect of some embodiments of the present invention, there is provided a method for treating a disease or disorder characterized by protease activity or, in some embodiments, elevated cysteine protease activity, in a subject in need thereof, the method comprising administering to the subject a composition comprising NUP, wherein the NUP is a fraction of a Nymphaeaceae extract comprising a compound selected from:

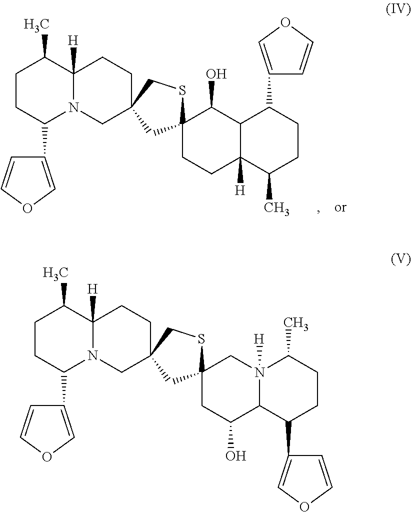

In some embodiments, the disease or disorder is selected from the group consisting of: an inflammatory disease, a neurological disease, a parasitic disease, and a cancer.

In some embodiments, the Nymphaeaceae is Nuphar lutea.

In some embodiments, the cysteine protease is papain.

According to an aspect of some embodiments of the present invention, there is provided a pharmaceutical composition for treating a disease or a disorder characterized by an elevated cysteine protease activity, the composition comprising one or more of the disclosed compounds.

In some embodiments, the disclosed composition is packaged in a packaging material and identified in print, in or on the packaging material, for use for treating a disease or disorder characterized by protease activity or, in some embodiments, by elevated cysteine protease activity.

In some embodiments, the pharmaceutical composition is for parenteral, mucosal, nasal or oral administration.

According to an aspect of some embodiments of the present invention, there is provided a use of one or more of the disclosed compounds or a pharmaceutically acceptable salt thereof in the manufacture of a medicament for the treatment of a disease or disorder characterized by protease activity or, in some embodiments, by elevated cysteine protease activity.

In some embodiments, one or more of the disclosed compounds or a pharmaceutically acceptable salt thereof, are for treating a disease or a disorder characterized by an elevated cysteine protease activity.

According to an aspect of some embodiments of the present invention, there is provided method of detecting a cysteine protease from a biological sample, the method comprising the steps of:

a) contacting a sample comprising a cysteine protease with one or more compounds of selected from:

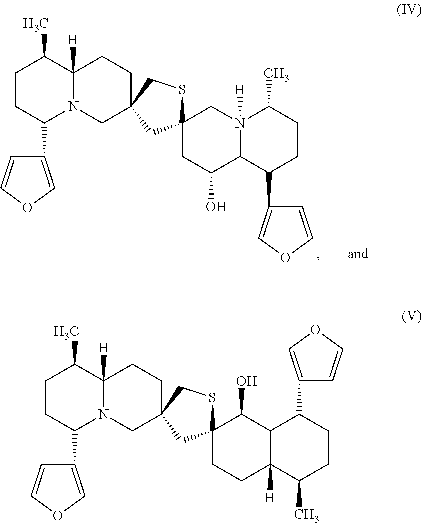

or a compound represented by Formula I as described hereinabove;

b) subjecting the sample to conditions that allow for selective binding of the cysteine protease with the compound.

In some embodiments, the compound is further conjugated to one or more agents selected from the group consisting of: a chelating agent, a complexing agent and an epitope tag.

In some embodiments, the method further comprises a step of separating the agent from the sample, thereby purifying and/or isolating the cysteine protease from the biological sample.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a bar graph showing inhibition of papain activity by a compound represented by compound III, as described hereinbelow; and

FIG. 2 presents graphs showing the effect of various nupharidine fractions on trypsin reaction rate.

### DETAILED DESCRIPTION OF THE INVENTION

The present invention, in some embodiments thereof, relates to synthetic compounds, compounds derived from a plant extract, and compositions comprising same, for treating a disease or a disorder characterized by protease activity, for example, elevated protease activity. In some embodiments, the protease refers to cysteine protease. In some embodiments, the pharmaceutical composition comprises NUP, wherein the NUP is a fraction of a Nymphaeaceae. In some embodiments, the pharmaceutical composition comprises an active fraction of a nupharidine. In some embodiments, the pharmaceutical composition comprises a nupharidine from NUP. In some embodiments, the pharmaceutical composition comprises a nupharidine not derived from NUP.

In some embodiments, there is provided herein a pharmaceutical composition for treating a disease or a disorder characterized by protease activity, or, in some embodiments, by elevated cysteine protease activity. In some embodiments, the composition comprises a compound having the general formula I:

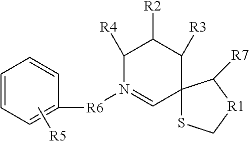

wherein:

R1 is CH₂, C₂-C₄ alkyl or is represented by the structure of Formula II:

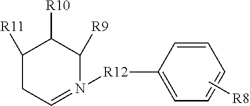

such that Formula I is in the form of Formula Ia:

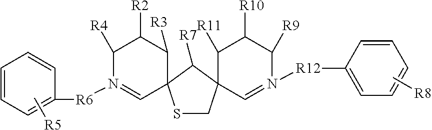

and wherein:

R2, R3, R4, R7, R9, R10, R11 are independently selected at each occurrence from hydrogen or C1-C8-alkyl optionally substituted by hydroxyl;

R6 and R12 are independently C1-C8 alkyl;

each R5 and R8 represents 0, 1, 2, 3, 4, or 5 substitutents independently selected at each occurrence from the group consisting of: hydroxy, halogen, cyano, C₁-C₄ alkyl, C₁-C₄ alkoxy, C₁-C₄ haloalkyl, C₁-C₄ haloalkoxy, —SO₂—R¹³, —P(O)(OR¹⁴)(OR¹⁵), —C(O)—NR¹⁴R¹⁵, —N(CH₃)—CO—O—(C₁-C₄) alkyl, —NH—CO—O—(C₁-C₄) alkyl, —NH—CO—(C₁-C₄)alkyl, —N(CH₃)—CO—(C₁-C₄) alkyl, —NH—(CH₂)₂—OH;

R¹³ is selected from: C₁-C₄ alkyl, NH₂, or —(CH₂)₂—OH;

R¹⁴ is selected from: hydrogen or C₁-C₄ alkyl; and

R¹⁵ is selected from: hydrogen, C₁-C₄ alkyl, —(CH₂)₂—OH, —(CH₂)₂—O—CH₃, —(CH₂)₃—OH, —(CH₂)₃—O—CH₃, 3-oxetany.

In some embodiments, R1 is CH₂, R5 represents 0 substituent, and R2, R3, R4, and R7 are each hydrogens.

In some embodiments, the pharmaceutical composition comprises a compound represented by the structure of formula III (“compound III”):

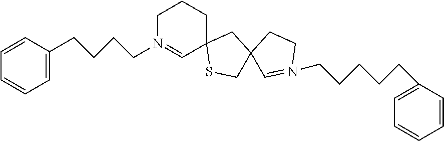

Further exemplary embodiments of compounds having the general formula I are described in the Examples section below.

In some embodiments, the NUP comprises thioalkaloids. In some embodiments, NUP is a purified plant extract composition comprising a thioalkaloid.

In some embodiments, the thioalkaloid is a dimeric sesquiterpene thioalkaloid. In some embodiments, the disclosed composition comprises thionupharidines and thionuphlutidines.

In some embodiments, NUP is isolated from Nymphaeaceae. In some embodiments, the NUP is a fraction of a Nymphaeaceae extract. In one embodiment, NUP comprises a compound selected from (compound IV):

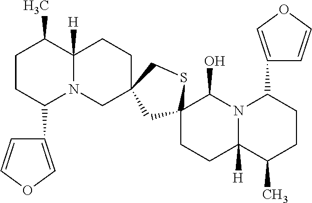

or (compound V):

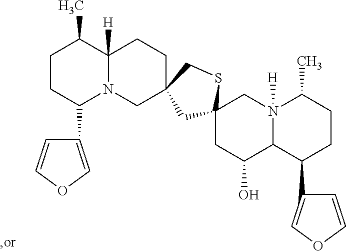

In some embodiments, the amount of one or more of the compounds disclosed herein required for suppressing the protease (e.g., cysteine protease) activity is 1 to 100 mg/kg per day. In some embodiments, the amount is 1 to 50 mg/kg per day. In some embodiments, the amount is 1 to 10 mg/kg per day. In some embodiments, the amount is 5 to 30 mg/kg per day. In some embodiments, the amount is 7 to 25 mg/kg per day.

The dosage may be e.g., at least once a day for at least one day, at least once a day for at least two days, at least once a day for at least three days, at least once a day for at least four days, or at least once a day for at least one week or more.

In some embodiments, the dosage is in a solid unit form. In some embodiments, the dosage is in a liquid form.

In some embodiments, solid unit dosage forms (e.g., tablets or capsules) may be manufactured by a variety of different methods, as are well known in the art, including a direct compression using a tablet punch. As an alternative to direct compression, the active ingredient and excipients may be combined by dry blending, and then subjected to dry granulation prior to tablet compression. A further alternative method is to utilize wet granulation, in which at least some of the excipients, together with the active ingredient, are blended and then further mixed in the presence of a granulation liquid. Following aggregation of the various powders, the aggregates (i.e. granules) are then sized by screening or milling, dried and used to produce a tablet. The tablet may be finally coated. Solid formulation blends for loading into capsules (such as soft gelatin capsules) may be prepared by dry blending, or by wet or dry granulation prior to being introduced into the capsules.

Further embodiments of this aspect of the present embodiments are included hereinbelow, under “Pharmaceutical Composition of the Compounds”, and form an integral part of embodiments relating to pharmaceutical composition.

In some embodiments, the disease or disorder is selected from, without being limited thereto, an inflammatory disease, a neurological disease, parasitic disease, and a cancer.

In some embodiments, the compounds disclosed herein or any combination thereof suppress cysteine protease activity in a subject.

In some embodiments, the inflammation is related to oral inflammatory syndrome. By “oral” it is meant to include, without limitation, lip, tongue, and gums.

In some embodiments, one or more compounds described hereinthroughout are characterized by increased binding affinity to a cysteine-protease.

In some embodiments, the terms “cysteine protease”, or “cysteine peptidase”, refer to the family of peptidases which have a common catalytic mechanism that involves a nucleophilic cysteine thiol in a catalytic triad.

Without being bound by any particular mechanism, the first step is deprotonation of a thiol in the enzyme's active site by an adjacent amino acid with a basic side chain, usually a histidine residue. Cysteine proteases may have characteristic molecular topologies, which can be seen not only in their three-dimensional structures, but commonly also in the two-dimensional structures.

In some embodiments, the term “binding affinity”, as used herein, refers to the strength of the sum total of non-covalent interactions between a single binding site of a molecule and its binding partner.

As used herein cysteine residue may be located in a peptide or in a protein, and/or be incorporated or immobilized in a substrate. In some embodiments, the substrate is a part of a kit, e.g., a diagnostic kit as described hereinbelow.

In some embodiments, the terms “polypeptide” and “peptide” encompass an amino acid sequence of any length including full-length proteins or portions thereof, wherein the amino acid residues are linked by covalent peptide bonds. Generally, an amino acid sequence of 50 amino acids and more are referred to herein as “polypeptide” or “protein”, and an amino acid sequence of less than 50 amino acids is referred to herein as “peptide”.

In some embodiments, the term “peptide” as used herein encompasses also peptoids and semipeptoids which are peptide analogs, which may have, for example, modifications rendering the peptides more stable while in a body or more capable of penetrating into cells. Such modifications include, but are not limited to, N-terminus modification, C-terminus modification, peptide bond modification, including, without being limited thereto, CH₂—NH, CH₂—S, CH₂—S═O, O═C—NH, CH₂—O, CH₂—CH₂, S═C—NH, CH═CH or CF═CH, backbone modifications, and residue modification.

As used herein, the phrase “amino acid residue”, which is also referred to herein, interchangeably, as “amino acid”, describes an amino acid unit within a polypeptide chain. The amino acid residues within the peptides described herein can be either natural (naturally-occurring) or modified (non-naturally occurring) amino acid residues, as these phrases are defined hereinafter.

As used herein, the phrase “natural amino acid residue” describes an amino acid residue, as this term is defined hereinabove, which includes one of the twenty amino acids found in nature.

Accordingly, in some embodiments, the term “amino acid” used above is understood to include the 20 naturally occurring amino acids; those amino acids often modified post-translationally in vivo, including, for example, hydroxyproline, phosphoserine, and phosphothreonine, and other unusual amino acids including, but not limited to, 2-aminoadipic acid, hydroxylysine, isodesmosine, nor-valine, nor-leucine and omithine.

In some embodiments, the term “amino acid” includes D- and/or L-amino acids. In some embodiments, the term “oligopeptide” refers to a series of amino acids linked by peptide bonds.

Suitable sequences of amino acids may be chosen according to the teachings of Cordingley et al, J. Biol Chem., 265(16), 9062-9066 (1990).

In some embodiments, various amino acids may be screened as follows: a desirable protease, such as the 3C picomavirus protease is immobilized to a solid support. Candidate sequences are brought into contact with the immobilized protease. Those residues which bind to the immobilized proteases may be chosen as candidate sequences.

As known in the art, cysteine proteases are divided into clans (proteins which are evolutionary related), and further sub-divided into families, on the basis of the architecture of their catalytic dyad or triad. The cysteine protease families include, but are not limited to, papain, cathepsins, cruzain, caspases, legumain, gingipains, clostripain, calpains.

In exemplary embodiments, cysteine-proteases are selected from caspase, calpain and adenine.

In some embodiments, the disclosed pharmaceutical compositions are suitable for the treatment of diseases which their manifestation is dependent on cysteine proteases of the CA Clan. Proteases of Clan CA include, inter alia, vital mammalian enzymes (e.g., papain, cathepsins), i.e. peptides of which normal activity is essential.

In some embodiments, the disclosed pharmaceutical compositions are suitable for the treatment of diseases which their manifestation is dependent on cysteine proteases which involve cysteine proteases of the CB Clan (e.g., families C3, C4, C24, C30, C37 and C38), for examples, human rhinovirus, 3C protease, aspartate proteases, serine proteases, metallo-proteases. In some embodiments, the inhibition is of “picomavirus 3C-like cysteine proteases”, which are cysteine protease having an active site similar to the active site of the 3C protease having a catalytic dyad of Histidine and Cysteine.

In some embodiments, the disclosed pharmaceutical compositions are suitable for the treatment of diseases which their manifestation is dependent on cysteine proteases of Clan CC. Clan CC includes 16 families of papain-like viral peptidases (C6-C9, C16, C21, C23, C27-29, C31-C36), comprising catalytic dyad of histidine and cysteine.

In some embodiments, the disclosed pharmaceutical compositions are suitable for the treatment of diseases which their manifestations involve cysteine proteases of the CD Clan (e.g., families C11, C13, C14, and C25).

In some embodiments, the disclosed pharmaceutical compositions are suitable for the treatment of diseases which manifestation is dependent on cysteine proteases CE clan.

By “diseases which their manifestation is dependent on cysteine proteases” it is meant to refer to a disease which can be treated, prevented, alleviated or cured by inhibition of the corresponding cysteine proteases, e.g., of the CB Clan, the CD Clan, the CE clan as described above.

In some embodiments, the pharmaceutical compositions of the invention are for the treatment of viral infections and of diseases wherein excessive apoptosis is implicated and/or wherein apoptosis is desired to be reduced.

In some embodiments, the disease is picomaviral infection, neurodegenerative disease, or a certain cardiovascular disease.

As noted hereinabove, in some embodiments, the herein disclosed pharmaceutical compositions are suitable also for the treatment of diseases manifested by the activity of the cysteine proteases of the CB clan. Therefore, in some embodiments, the disclosed pharmaceutical compositions are suitable for the treatment of: common colds, allergic rhinitis, poliomyelitis, hepatitis-A, encephalitis, meningitis, hand foot-and-mouth disease, encephalomyocarditis, summer flu (enteroviral upper respiratory infection), asthma, various allergies, myocarditis, acute hemorrhagic conjunctivitis, disseminated neonatal infection and Borhnolm's disease.

In some embodiments, the disease refers to a parasitic disease. Non-limiting exemplary parasites known to utilize cysteine proteases in their life cycle include Trypanosoma cruzi, Trypanosoma Brucei [trypanosomiasis (African sleeping sickness, Chagas disease)], Leishmania mexicana, Leishmania pifanoi, Leishmania major (leishmaniasis), Schistosoma mansoni (schistosomiasis), Onchocerca volvulus [onchocerciasis (river blindness)] Brugia pahangi, Entamoeba histolytica, Giardia lambia, the helminths, Haemonchus contortus and Fasciola hepatica, as well as helminths of the genera Spirometra, Trichinella, Necator and Ascaris, and protozoa of the genera Cryptosporidium, Eimeria, Toxoplasma and Naegleria. The present pharmaceutical compositions allows treatment of diseases caused by infection by these parasites by inhibiting protease (for example, cysteine proteasee of the papain superfamily) by administering to a patient in need thereof.

Without being bound by any particular theory or mechanism, one or more compounds of the disclosed pharmaceutical compositions selectively bind to the proteases, and may compete with the substrates for proteases. For example, this competition serves to inhibit viral maturation and thus to inhibit disease progression in vivo.

As noted hereinabove, in some embodiments, the herein disclosed pharmaceutical compositions are suitable also for the treatment of diseases manifested by the activity of the cysteine proteases of the CD clan, i.e. apoptosis-involved diseases, which includes activation (as in cancer), as well as inhibition (as in neurodegenerative diseases) of apoptosis.

In some embodiments, the pharmaceutical compositions of the present invention are also suitable for the treatment of adenoviral-involved diseases.

It will be recognized that these embodiments are susceptible to various modifications and alternative forms well known to those of skill in the art.

### Definitions

As used herein, the term “alkyl” describes an aliphatic hydrocarbon including straight chain and branched chain groups. Preferably, the alkyl group has 21 to 100 carbon atoms, and more preferably 21-50 carbon atoms. Whenever a numerical range; e.g., “21-100”, is stated herein, it implies that the group, in this case the alkyl group, may contain 21 carbon atom, 22 carbon atoms, 23 carbon atoms, etc., up to and including 100 carbon atoms. In the context of the present invention, a “long alkyl” is an alkyl having at least 20 carbon atoms in its main chain (the longest path of continuous covalently attached atoms). A short alkyl therefore has 20 or less main-chain carbons. The alkyl can be substituted or unsubstituted, as defined herein

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an —O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, alkoxy, nitro, amine, hydroxyl, thiol, thioalkoxy, thiohydroxy, carboxy, amide, aryl and aryloxy, depending on the substituted group and its position in the molecule. Additional substituents are also contemplated

The term “halide”, “halogen” or “halo” describes fluorine, chlorine, bromine or iodine.

The term “haloalkyl” describes an alkyl group as defined herein, further substituted by one or more halide(s).

The term “haloalkoxy” describes an alkoxy group as defined herein, further substituted by one or more halide(s).

The term “hydroxyl” or “hydroxy” describes a —OH group.

The term “thiohydroxy” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amine” describes a —NR′R″ group, with R′ and R″ as described herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

The term “carboxy” or “carboxylate” describes a —C(═O)—OR′ group, where R′ is hydrogen, alkyl, cycloalkyl, alkenyl, aryl, heteroaryl (bonded through a ring carbon) or heteroalicyclic (bonded through a ring carbon) as defined herein.

The term “carbonyl” describes a —C(═O)—R′ group, where R′ is as defined hereinabove.

The above-terms also encompass thio-derivatives thereof (thiocarboxy and thiocarbonyl).

The term “thiocarbonyl” describes a —C(═S)—R′ group, where R′ is as defined hereinabove.

A “thiocarboxy” group describes a —C(═S)—OR′ group, where R′ is as defined herein.

A “sulfinyl” group describes an —S(═O)—R′ group, where R′ is as defined herein.

A “sulfonyl” or “sulfonate” group describes an —S(═O)₂—R′ group, where Rx is as defined herein.

A “carbamyl” or “carbamate” group describes an —OC(═O)—NR′R″ group, where R′ is as defined herein and R″ is as defined for R′.

A “nitro” group refers to a —NO₂ group.

A “cyano” or “nitrile” group refers to a —C≡N group.

As used herein, the term “azide” refers to a —N₃ group.

The term “sulfonamide” refers to a —S(═O)₂—NR′R″ group, with R′ and R″ as defined herein.

The term “phosphonyl” or “phosphonate” describes an —O—P(═O)(OR′)₂ group, with R′ as defined hereinabove.

The term “phosphinyl” describes a —PR′R″ group, with R′ and R″ as defined hereinabove.

The term “alkaryl” describes an alkyl, as defined herein, which substituted by an aryl, as described herein. An exemplary alkaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

As used herein, the terms “halo” and “halide”, which are referred to herein interchangeably, describe an atom of a halogen, that is fluorine, chlorine, bromine or iodine, also referred to herein as fluoride, chloride, bromide and iodide.

The term “haloalkyl” describes an alkyl group as defined above, further substituted by one or more halide(s).

Pharmaceutical Composition Comprising the Disclosed Compounds:

According to an aspect of embodiments of the invention there is provided a pharmaceutical composition comprising one or more compounds as described herein and a pharmaceutically acceptable carrier.

According to some embodiments of the invention, the composition is being packaged in a packaging material and identified in print, in or on the packaging material, for use in the treatment of a medical condition associated with any disease, medical condition, or disorder as described hereinthroughout.

According to an aspect of embodiments of the invention there is provided a method of treating a medical condition associated with any disease, medical condition, or disorder as described hareinthroughout in a subject in need thereof, the method comprising administering to the subject a therapeutically effective amount of the compound or composition as described herein.

The term “subject” (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters, guinea pigs, and so on.

In some embodiments, the subject is a human. In another embodiment, the subject is a human suffering from a disease associated with an elevated cysteine protease activity, as described hereinbelow.

According to an aspect of embodiments of the invention there is provided a use of any one of the compound as described herein as a medicament.

According to an aspect of embodiments of the invention there is provided a use of any one of the compound as described herein in the manufacture of a medicament for treating a medical condition associated with any disease, medical condition, or disorder associated with an elevated cysteine protease activity.

The compounds described hereinabove may be administered or otherwise utilized either as is, or as a pharmaceutically acceptable salt, enantiomer, diastereomer, solvate, hydrate or a prodrug thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound. The neutral forms of the compounds may be regenerated by contacting the salt with a base or acid and isolating the parent compound in a conventional manner. The parent form of the compound differs from the various salt forms in certain physical properties, such as solubility in polar solvents, but otherwise the salts are equivalent to the parent form of the compound for the purposes of the present invention. The phrase “pharmaceutically acceptable salts” is meant to encompass salts of the active compounds which are prepared with relatively nontoxic acids or bases, depending on the particular substituents found on the compounds described herein.

Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compound as described herein to be converted into either base or acid addition salts.

In some embodiments, the neutral forms of the compounds described herein are regenerated by contacting the salt with a base or acid and isolating the parent compounds in a conventional manner. The parent form of the compounds differs from the various salt forms in certain physical properties, such as solubility in polar solvents, but otherwise the salts are equivalent to the parent form of the conjugate for the purposes of the present invention.

The term “prodrug” refers to an agent, which is converted into the active compound (the active parent drug) in vivo. Prodrugs are typically useful for facilitating the administration of the parent drug. The prodrug may also have improved solubility as compared with the parent drug in pharmaceutical compositions. Prodrugs are also often used to achieve a sustained release of the active compound in vivo.

In some embodiments, the compounds described herein possess asymmetric carbon atoms (optical centers) or double bonds; the racemates, diastereomers, geometric isomers and individual isomers are encompassed within the scope of the present invention.

As used herein and in the art, the term “enantiomer” describes a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems.

In some embodiments, the compounds described herein can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

According to another aspect of embodiments of the invention there is provided a pharmaceutical composition comprising, as an active ingredient, any of the compounds described herein and a pharmaceutically acceptable carrier

Accordingly, in methods and uses described herein, one or more of the compounds described herein can be provided to an individual either per se, or as part of a pharmaceutical composition where it is mixed with a pharmaceutically acceptable carrier.

In some embodiments, the “pharmaceutical composition” refers to a preparation of one or more of the compounds described herein (as active ingredient), or physiologically acceptable salts or prodrugs thereof, with other chemical components including, but not limited to, physiologically suitable carriers, excipients, lubricants, buffering agents, antibacterial agents, bulking agents (e.g., mannitol), antioxidants (e.g., ascorbic acid or sodium bisulfate), anti-inflammatory agents, anti-viral agents, chemotherapeutic agents, anti-histamines and the like.

In some embodiments, the purpose of a pharmaceutical composition is to facilitate administration of a compound to a subject. The term “active ingredient” refers to a compound, which is accountable for a biological effect.

The terms “physiologically acceptable carrier” and “pharmaceutically acceptable carrier”, which may be interchangeably used, refer to a carrier or a diluent that does not cause significant irritation to an organism and does not abrogate the biological activity and properties of the administered compound.

Herein the term “excipient” refers to an inert substance added to a pharmaceutical composition to further facilitate administration of a drug. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

In some embodiments, pharmaceutical compositions for use in accordance with the present invention thus may be formulated in conventional manner using one or more pharmaceutically acceptable carriers comprising excipients and auxiliaries, which facilitate processing of the compounds into preparations which can be used pharmaceutically. Proper formulation is dependent upon the route of administration chosen. The dosage, as described and specified herein, may vary depending upon the dosage form employed and the route of administration utilized. The exact formulation, route of administration and dosage can be chosen by the individual physician in view of the patient's condition (see e.g., Fingl et al., 1975, in “The Pharmacological Basis of Therapeutics”, Ch. 1 p. 1).

In some embodiments, the pharmaceutical composition may be formulated for administration in either one or more of routes depending on whether local or systemic treatment or administration is of choice, and on the area to be treated. As further described hereinthroughout, administration may be done orally, by inhalation, or parenterally, for example by intravenous drip or intraperitoneal, subcutaneous, intramuscular or intravenous injection, or topically (including ophtalmically, vaginally, rectally, intranasally).

Formulations for topical administration may include but are not limited to lotions, ointments, gels, creams, suppositories, drops, liquids, sprays and powders. Conventional pharmaceutical carriers, aqueous, powder or oily bases, thickeners and the like may be necessary or desirable.

Compositions for oral administration include powders or granules, suspensions or solutions in water or non-aqueous media, sachets, pills, caplets, capsules or tablets. Thickeners, diluents, flavorings, dispersing aids, emulsifiers or binders may be desirable.

Formulations for parenteral administration may include, but are not limited to, sterile solutions which may also contain buffers, diluents and other suitable additives. Slow release compositions are envisaged for treatment.

The amount of a composition to be administered will, of course, be dependent on the subject being treated, the severity of the affliction, the manner of administration, the judgment of the prescribing physician, etc.

The pharmaceutical composition may further comprise additional pharmaceutically active or inactive agents such as, but not limited to, an antibacterial agent, an antioxidant, a buffering agent, a bulking agent, a surfactant, an anti-inflammatory agent, an anti-viral agent, a chemotherapeutic agent and anti-histamine.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Methods of Treatments

In some embodiments, there is provided a method for treating a disease or a disorder characterized by protease activity, e.g., cysteine protease activity (or elevated activity thereof) in a subject in need thereof.

In some embodiments, the term “elevated activity”, as used hereinthroughout, refers to an increase in the expression of endogenous genes coding for the corresponding protease. In some embodiments, the term “elevated activity” refers to an increase in the quantity of proteases in the cells. In some embodiments, the term “elevated activity” refers to an increase in the enzymatic activity of proteases in the cells.

By “increased” it is meant e.g., at least 0.1%, 1%, 5%, 10%, or 20%, higher compared to healthy subjects. For example, the term “increased activity” may refer to the quantity of proteases in the cells isolated from blood of a subject, being at least 0.1% higher than the quantity of proteases in the cells of a healthy subject.

In some embodiments, the method comprises administering to the subject a pharmaceutically effective amount of one or more of the herein disclosed compounds or compositions (e.g., compounds having one of the general “Formula I” described hereinabove, a composition comprising NUP, or the one or more compounds extracted from the fraction of the Nymphaeaceae ((e.g., compounds IV or V).

In some embodiments, the method may further comprise administrating a drug intended to treat a disease characterized by an elevated cysteine protease activity in a subject.

An elevated cysteine protease activity is involved in several disease states derived from, without limitation, an inflammation, viral and parasitic infections, a stroke, a cancer, neuronal cell death, arthritis, cardiovascular diseases, such as ischemic cardiac damage, neurodegenerative diseases such as, without limitation, Alzheimer, Parkinson and Huntington.

In some embodiments, a disease characterized by an elevated activity of the protease may be selected from, without limitation, abdominal aortic aneurysm (AAA), adult respiratory distress syndrome, septic shock, chronic obstructive pulmonary disease, pulmonary emphysema, and pulmonary hypertension.

Thus, in some embodiments, the method comprises a treatment of viral infection by administrating to a subject a pharmaceutically acceptable amount of the disclosed compound which has protease inhibitor activity, optionally together with a pharmaceutically acceptable carrier.

In some embodiments, the method enhances the efficacy of a composition comprising an anticancer drug. In some embodiments, the method for enhancing the efficacy yields a synergistic therapeutic, anti-cancer effect, which requires less amount of the anticancer drug.

In some embodiments, the method is for treating of an elevated cysteine protease activity by administrating to a subject a pharmaceutically acceptable amount of the disclosed compound (e.g., of formula (I)), optionally together with a pharmaceutically acceptable carrier.

A further aspect of the invention relates to the use of one or more compounds set forth hereinabove in the preparation of a medicament, e.g., for the treatment of a disease or disorder in a mammal, and more specifically, for the treatment of a disease or a disorder characterized by an elevated cysteine protease activity.

Diagnosis

The invention, in one aspect, also provides a method of purifying, isolating and/or immobilizing protease (e.g., cysteine protease) from a biological sample, the method comprising the steps of: a) contacting a sample comprising protease with any compound disclosed herein and b) subjecting the sample to conditions that allow for selective binding of the cysteine protease with said compound.

Depending on the choice of immobilization technique, the above-described method may comprise the additional step (step c) of combining the sample comprising the protease (e.g., cysteine protease) capturing agent with a solid phase capable of immobilizing the protease capturing agent, prior to any one of steps a, b, or c.

As used herein the term “capturing agent” may include any chelating agent water soluble metal chelate), complexing agent (i.e. water soluble metal complex), or epitope tag known in the art. If the immobilization step is done after step b), as will be understood, a technique is to be selected involving selective trapping under condition which does not affect other components of the biological sample. Hence, it will be appreciated that in certain embodiments of the method comprises immobilization of the protease capturing agent prior to step b.

As will be understood by those skilled in the art, immobilization of the cysteine protease capturing agent can be accomplished in various ways. In some embodiments, the cysteine protease capturing agent is immobilized using activated sepharose.

In some embodiments of the invention, the above method involves the use of a protease (e.g., cysteine protease) capturing agent that is conjugated/derivatized with a detection label as defined herein above, wherein the method comprises one or more additional steps of quantifying the binding of cysteine protease to the protease capturing agent.

As explained herein before the present protease (e.g., cysteine protease) agents bind their corresponding protease in a selective and highly irreversible manner, allowing for stringent washing conditions, which makes the present method highly effective.

The above method may be used in research concerning any biological process involving the action of protease (e.g., cysteine protease) and/or in diagnosing any condition or disease involving the action of protease.

Since, for example, the present cysteine protease capturing agents are capable of selective and highly irreversible binding of their corresponding cysteine protease, it is also envisaged that the cysteine protease capturing agents have utility as (competitive) protease inhibitors or antagonistic agents in various therapeutic methods. Typically, but not exclusively, such therapeutic methods are aimed at treating or preventing of a condition or disease, involving the action of a cysteine protease.

General

It is expected that during the life of a patent maturing from this application many relevant nupharidine based chemical structure and uses the same will be developed and the scope of the herein disclosed compounds is intended to include all such new compounds and technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find support in the following examples.

### EXAMPLES

Reference is now made to the following examples, which together with the above descriptions illustrate some embodiments of the invention in a non limiting fashion.

### Example 1

### Assays

Inhibition of Papain Activity by Compound III (Also Referred to as “6-61”):

The Protease Fluorescent Detection Kit of Sigma Aldrich was used for measurement of protease activity using fluorometry. In exemplary embodiments, the method is based on the proteolytic hydrolysis of a proprietary formulation of a FITC-labeled casein substrate. The reaction mixture contained in a final volume of 50 μL, 10 μg papain and various concentrations of the candidate inhibitor.

### Results

Inhibition of Papain Activity by Compound III:

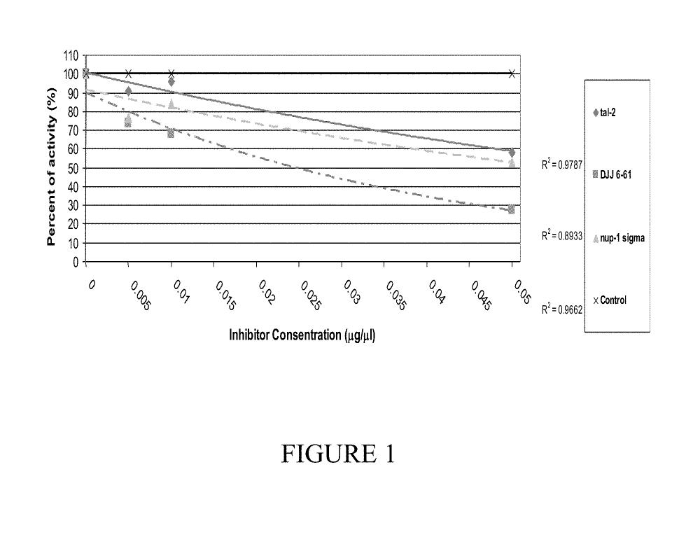

As shown in FIG. 1, compound III has demonstrated an inhibition of papain activity.

### Example 2

### Assays

In exemplary procedures, inhibition assays were performed for various inhibitors as listed in Table 1 hereinbelow.

The assays were performed in triplicates using transparent 96 well plates (167008; Nunc, Roskilde, Denmark). Reaction mixture 100 μl contained: 35 μl reaction buffer (0.1 M phosphate buffer, pH 6.5), 50 μl of Casein FITC (C3777-25MG; sigma), serially dissolved in reaction buffer to concentrations ranging from 10 μg/100 μl to 1.25 μg/100 μl in the final reaction. Inhibitors dissolved in DMSO were added in 5 μl aliquots at final concentrations in reaction mixture ranging from 0.05 μg/μl to 0.005 μg/μl. In control reaction 5 μl DMSO was added. Papain (P3125-100 mg; sigma) stock of 5 μg/100 μl dissolved in reaction buffer was added last in a 10 μl aliquot at a final concentration of 0.5 μg/100 μl reaction mixture, just before the fluorometric measurement. The data was collected for 2.5 h every 90 sec. Initial reaction velocity in reactions containing various substrate concentrations ranging from 1.25 to 10 μg/μl were used to calculate the IC50 values. Control reaction not containing enzyme was used as baseline.

### Results

Inhibition assays were performed in triplicates using 96 well plates transparent for Calpain, Trypsin, Papain, Cathepsin L and Cathepsin B (167008; Nunc, Roskilde, Denmark), or black for Cathepsin S, Cathepsin D and Insulyzin (ide) (Greiner 96 Polystyrol GRE96fb) according to manufacturers' instructions. Briefly, reaction mixture of 100 μl contained: 45 μl of enzyme in assay buffer, 5 μl of inhibitors diluted in DMSO. 50 μl substrate prepared in assay buffer according to manufacturers' instructions. The inhibitor was serially diluted in DMSO to concentrations from 0.0075 μg/μL to 0.0000075 μg/μL. 5 μl DMSO was added to control sample. Cathepsin substrate mix was added last just before starting the fluorometric measurement. Data was collected for 1.5 h at 60 sec intervals. Molarity of inhibitors were calculated based on assumed molecular weight of 526 daltons as a reference value for comparative evaluations.

The results have shown that a partially purified nupharidine fraction inhibited e.g., Papain, as shown in Table 1, showing the inhibition of papain by Nuphar Lutea purified fractions of nupharidines and synthetic derivatives.

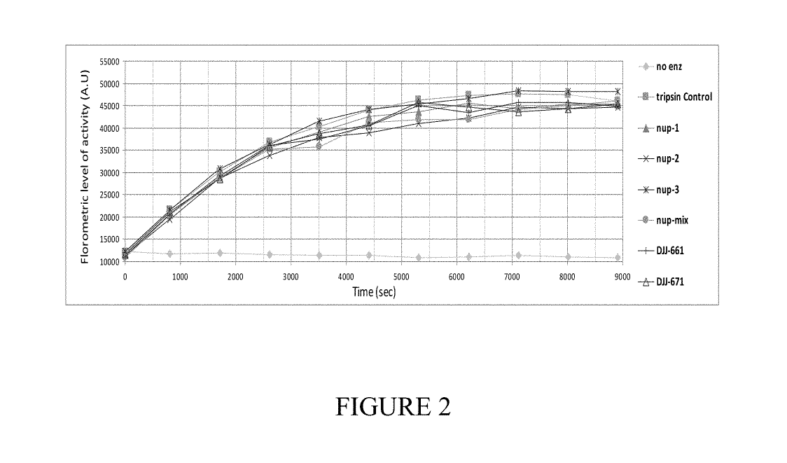

This type of inhibition was also found in purified nupharidines from leaf and rhizome of Nuphar lutea (Table 1 below shows results of inhibition of proteases by natural nupharidines of Nuphar lutea and a synthetic derivative). Furthermore, a synthetic compound with a “thiaspirane warhead”, designated as “6-61”, as well as another derivative “195-1” showed a similar inhibitory activity against papain. The specificity of the reaction was tested by treatment of trypsin, a serine protease with the above mentioned compounds and no inhibitory activity was found (as shown in FIG. 2).

TABLE 1

| | | | IC50 | IC50 | |
|---|---|---|---|---|---|
| Enzyme name | Substrate | Inhibitor | (Microgram/ml) | (Nanomolar) | Buffer used/Ex, Em |
| Human Cathepsin S | Mca- | Pond-6; partially purified | 0.0000219 | 0.04 | 50 mM NaOAc, |
| | RPKPVE- | nupharidines from | | | 250 mM NaCl, |
| | Nval- | Nuphar lutea leaves | | | 5 mM DTT pH 4.5 |
| | WRK(Dnp)- | Nup-3 sigma*; | 0.000041 | 0.08 | Ex/Em = 320- |
| | NH2(ES002) | 6 6′dihydroxybinupharidine | | | 405 nm |
| Human Cathepsin L | Ac-FR-AFC | Nup-3 sigma*; | 0.00014 | 0.266 | CTSL Assay |
| | | 6 6′dihydroxybinupharidine | | | Buffer |
| | | Nup-4 sigma; | 0.0000295 | 0.056 | Ex/Em = 400- |
| | | 6 hydroxybinupharidine | | | 505 nm |
| Human rCathepsin D | Mca-PLGL- | Nup-3 sigma*; | 0.000035 | 0.07 | 0.1M NaOAc, |
| | Dpa-AR-NH2 | 6 6′dihydroxybinupharidine | | | 0.2M NaCl, |
| | (ES001) | Nup-4 sigma; | 0.000168 | 0.32 | pH 3.5 |
| | | 6 hydroxybinupharidine | | | Ex/Em = 320- |
| | | Pond-6; partially purified | 0.000325 | 0.62 | 405 nm |
| | | nupharidines from | | | |
| | | Nuphar lutea leaves | | | |
| Human Insulyzin | Mca- | Nup-3 sigma*; | 0.000174 | 0.33 | 50 mM Tris |
| (ide) | RPPGFSAFK | 6 6′dihydroxybinupharidine | | | 1M NaCl |
| | (Dnp)-OH | Nup-4 sigma; | 0.00045 | 0.85 | pH 7.5 |
| | (ES005) | 6 hydroxybinupharidine | | | Ex/Em = 320- |
| | | Pond-6; partially purified | 0.00023 | 0.44 | 405 nm |
| | | nupharidines from | | | |
| | | Nuphar lutea leaves | | | |
| Human Cathepsin B | Casein FITC | Nup-3 sigma*; | 0.0115 | 21.8 | 0.3M potassium |
| | | 6 6′dihydroxybinupharidine | | | chloride, |
| | | Nup-4 sigma; | 0.0155 | 29.4 | 0.1 mM EDTA, |
| | | 6 hydroxybinupharidine | | | and 3 mM DTT |
| | | | | | pH 6.5 |
| | | | | | Ex/Em = 485- |
| | | | | | 535 nm |
| Papain latex | Casein FITC | Nup-1 sigma; advanced | 0.056 | 105.82 | 0.3M potassium |
| | | purified nupharidines | | | chloride, |
| | | fraction | | | 0.1 mM EDTA, |
| | | Nup-2 sigma; advanced | 0.038 | 72.71 | and 3 mM DTT |
| | | purified nupharidines | | | pH 6.5 |
| | | fraction | | | Ex/Em = 485- |
| | | “6-61”; synthetic | 0.017 | 23.6 | 535 nm |
| | | thiaspirane (Ryan) | | | |
| | | “195-1”; synthetic | 0.056 | 78.46 | |
| | | thiasprane | | | |
| | | Pond-6; partially purified | 0.028 | 53.6 | |
| | | nupharidines from | | | |
| | | Nuphar lutea leaves | | | |

| | | | | |
|---|---|---|---|---|
| Trypsin | Casein FITC | “6-61”; synthetic | no inhibition | 0.3M potassium |
| | | thiaspirane (Ryan) | | chloride, |
| | | Nup-1 sigma; advanced | no inhibition | 0.1 mM EDTA, |
| | | purified nupharidines | | and 3 mM DTT |
| | | fraction | | pH 6.5 |
| | | Nup-2 sigma; advanced | no inhibition | Ex/Em = 485- |
| | | purified nupharidines | | 535 nm |
| | | fraction | | |
| | | Nup-3 sigma*; | no inhibition | |
| | | 6 6′dihydroxybinupharidine | | |
| | | Mixed Nup-1 + Nup 2 | no inhibition | |
| Calpain | Casein FITC | “6-61”; synthetic | no inhibition | 0.3M potassium |
| | | thiaspirane (Ryan) | | chloride, |
| | | Nup-1 sigma; advanced | no inhibition | 0.1 mM EDTA, |
| | | purified nupharidines | | and 3 mM |
| | | fraction | | DTT pH 6.5 |
| | | Nup-2 sigma; advanced | no inhibition | 2 mM CaCl₂ |
| | | purified nupharidines | | Ex/Em = 485- |
| | | fraction | | 535 nm |
| | | Nup-3 sigma*; | no inhibition | |
| | | 6 6′dihydroxybinupharidine | | |

“Tal 2” refers to a partially purified fraction, which contains mainly thio binuhatidine and thio binuphlutine.
*The structures of the Sigma compounds are quite pure, so are nup-L1 and L2 from 2 batches of preparation Nup-1 and -2, respectively, which may comprise one or two of the above-mentioned compounds. Nup-3 is a purified nupharidine from the rhizome.

The compounds are designated as follows:

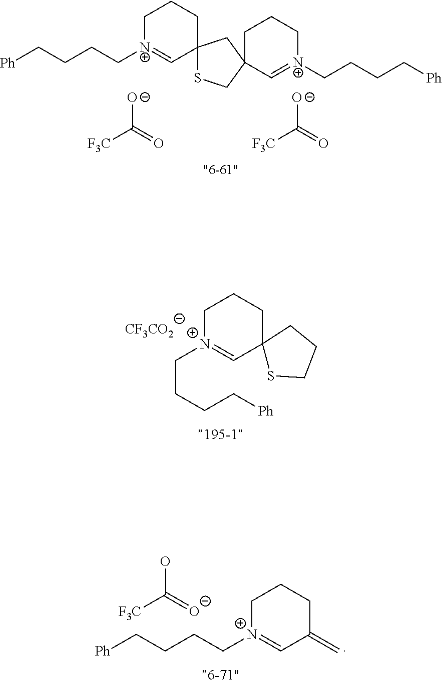

Taken together, it is shown that compound III has anti-proteolytic activity against cys-proteases as well as against other proteases with a sensitive nucleophile towards the above mentioned compounds, at their active site.

These results open new venues for testing the nupharidine-based compounds as therapeutic agents in medicinal conditions where proteases e.g., cys-proteases are involved.

The specificity of nupharidines from Nuphar lutea has been demonstrated for partially purified extracts as well as pure fractions (Sigma) towards papain e.g., cysteine-25 of papain, a cysteine protease and not towards e.g., calpain, and trypsin, a serine protease.

Without being bound by any particular theory or mechanism, the results supports the hypothesis concerning the mechanism underlying the action of the compound via “Michael addition” (i.e. electrophilic attack on a nucleophile at the active site of an enzyme) and opens a whole new potential for medicinal uses of this compound as an inhibitor of proteases with a sensitive nucleophile e.g., cys-proteases.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed:

1. A method for treating a disease or a disorder characterized by protease activity in a subject in need thereof, comprising administering to said subject a pharmaceutically effective amount of a compound having the general formula I: 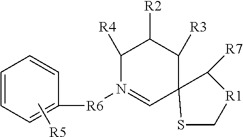
wherein:
R1 is CH₂, C₂-C₄ alkyl or is represented by the structure of Formula II: 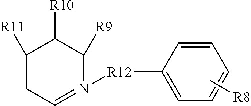
such that Formula I is in the form of Formula Ia: 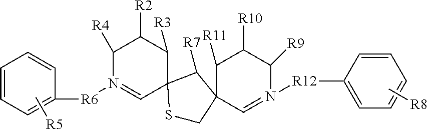
wherein:
R2, R3, R4, R7, R9, R10, R11 are independently selected at each occurrence from hydrogen or C1-C8-alkyl optionally substituted by hydroxyl;
R6 and R12 are independently C1-C8 alkyl;
each R5 and R8 represents 0, 1, 2, 3, 4, or 5 substituents independently selected at each occurrence from the group consisting of: hydroxy, halogen, cyano, C₁-C₄ alkyl, C₁-C₄ alkoxy, C₁-C₄ haloalkyl, C₁-C₄ haloalkoxy, —SO₂—R¹³, —P(O)(OR¹⁴)(OR¹⁵), —C(O)—NR¹⁴R¹⁵, —N(CH₃)—CO—O—(C₁-C₄) alkyl, —NH—CO—O—(C₁-C₄) alkyl, —NH—CO—(C₁-C₄)alkyl, —N(CH₃)—CO—(C₁-C₄) alkyl, —NH—(CH₂)₂—OH;
R¹³ is selected from: C₁-C₄ alkyl, NH₂, or —(CH₂)₂—OH;
R¹⁴ is selected from: hydrogen or C₁-C₄ alkyl; and
R¹⁵ is selected from: hydrogen, C₁-C₄ alkyl, —(CH₂)₂—OH, —(CH₂)₂—O—CH₃, —(CH₂)₃—OH, —(CH₂)₃—O—CH₃, 3-oxetany.

2. The method of claim 1, wherein R1 is CH₂, R5 represents 0 substituent, and R2, R3, R4, and R7 are each hydrogen.

3. The method of claim 1, wherein said compound is represented by the structure of formula III:

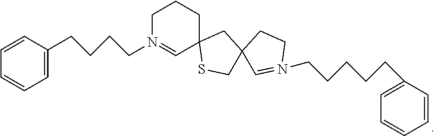

4. A method for treating a disease or disorder characterized by protease activity in a subject in need thereof, the method comprising administering to said subject a composition comprising a compound selected from compound IV: 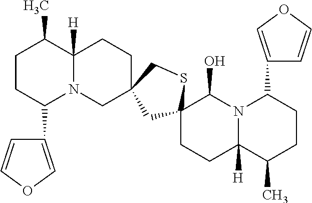
and compound V:

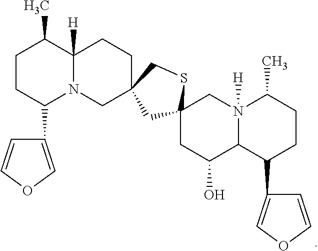

5-13. (canceled)

14. A method of detecting a cysteine protease in a sample comprising the steps of:
a) contacting a sample comprising said cysteine protease with one or more compounds of selected from compound IV: 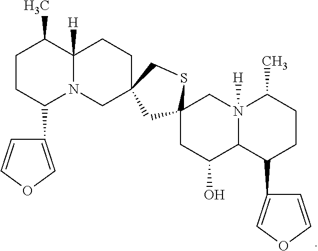
and compound V 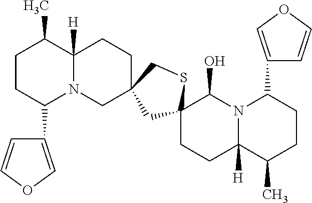
or a compound represented by Formula I: 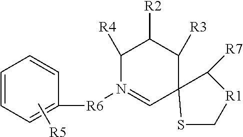
b) subjecting the sample to a condition that allows for selective binding of the cysteine protease with said compound,
wherein:
R1 is CH₂, C₂-C₄ alkyl or is represented by the structure of Formula II: 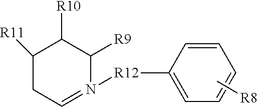
such that Formula I is in the form of Formula Ia: 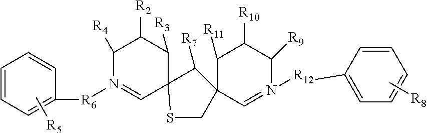
wherein:
R2, R3, R4, R7, R9, R10, R11 are independently selected at each occurrence from hydrogen or C1-C8-alkyl optionally substituted by hydroxyl;
R6 and R12 are independently C1-C8 alkyl;
each R5 and R8 represents 0, 1, 2, 3, 4, or 5 substituents independently selected at each occurrence from the group consisting of: hydroxy, halogen, cyano, C₁-C₄ alkyl, C₁-C₄ alkoxy, C₁-C₄ haloalkyl, C₁-C₄ haloalkoxy, —SO₂—R¹³, —P(O)(OR¹⁴)(OR¹⁵), —C(O)—NR¹⁴R¹⁵, —N(CH₃)—CO—O—(C₁-C₄) alkyl, —NH—CO—O—(C₁-C₄) alkyl, —NH—CO—(C₁-C₄)alkyl, —N(CH₃)—CO—(C₁-C₄) alkyl, —NH—(CH₂)₂—OH;
R¹³ is selected from: C₁-C₄ alkyl, NH₂, or —(CH₂)₂—OH;
R¹⁴ is selected from: hydrogen or C₁-C₄ alkyl; and
R¹⁵ is selected from: hydrogen, C₁-C₄ alkyl, —(CH₂)₂—OH, —(CH₂)₂—O—CH₃, —(CH₂)₃—OH, —(CH₂)₃—O—CH₃, 3-oxetany.

15. The method of claim 14, wherein said compound is further conjugated to one or more agents selected from the group consisting of: a chelating agent, a complexing agent and an epitope tag.

16. The method of claim 14, further comprising a step of separating said agent from said sample, thereby purifying and/or isolating said cysteine protease from said sample.
